(19) United States
(12) Patent Application Publication
(10) Pub. No.: US 2008/0171905 A1
(43) Pub. Date: Jul. 17, 2008

(54) Transvaginal Delivery of Bulking Masses Adjacent the Urethra to Alleviate Female Incontinence

(75) Inventor: Kimberly A. Anderson, Eagan, MN (US)

Correspondence Address: AMS RESEARCH CORPORATION, 10700 BREN ROAD WEST, MINNETONKA, MN, 55343, US

(73) Assignee: AMS RESEARCH CORPORATION, Minnetonka, MN (US)

(21) Appl. No.: 11/624,086

(22) Filed: Jan. 17, 2007

(51) Int. Cl. A61F 2/00; A61M 31/00

(52) U.S. Cl. 600/29; 604/515

(57) ABSTRACT

Transvaginal delivery instrumentation and methods are disclosed for delivering a biocompatible bulking mass transvaginally into a target tissue mass between the vaginal and urethral walls to constrict the urethra to alleviate female incontinence. Delivery is effected by urging a selected portion of the tissue structure between the vaginal and urethral walls into the vaginal cavity as a target structure, and passing a bulking mass from the vaginal cavity through the vaginal wall and into the target structure. Particular transvaginal delivery instrumentation comprises a vaginal and urethral probes adapted to be introduced into the vagina and urethra and bulking mass delivery instruments adapted to be introduced through a vaginal probe lumen and port into the target tissue structure.

### FIELD

The present invention relates to the field of treating female incontinence and particularly to a method of and apparatus for transvaginally delivering a bolus of bulking agent or an inflatable balloon (collectively referred to herein as a bulking mass) into a target tissue structure between the vaginal and urethral walls.

### BACKGROUND

As set forth in commonly assigned U.S. Pat. No. 6,964,699, urinary incontinence is a significant clinical problem and a major source of disability and dependency. The most frequently occurring types of urinary incontinence are stress incontinence, urge incontinence, overflow incontinence, and mixed incontinence.

Stress incontinence is a common form of incontinence in women. Intra-abdominal pressure exceeds urethral pressure upon coughing, sneezing, laughing, lifting, or like activity, causing leakage of urine. Physical changes associated with pregnancy, childbirth, and menopause, for example, are known to cause stress incontinence.

Urge incontinence occurs when a patient loses urine while suddenly feeling the urge to urinate. The patient is unable to inhibit the flow of urine long enough to reach the toilet. Inappropriate bladder contractions are the most common cause of urge incontinence, and may occur in connection with central nervous system lesions, urinary infection, or bladder tumors, to name several examples.

Overflow incontinence occurs when the bladder is unable to empty normally. Weak bladder muscles, caused e.g. by nerve damage from diabetes, or a blocked urethra, caused e.g. by tumors or urinary stones, are among the more common causes of overflow incontinence. Frequency or urgency involves the need or urge to urinate on an excessively frequent or habitual basis. Combinations of these and other types of incontinence, e.g. stress incontinence and urge incontinence, are often called mixed incontinence.

Many options are available to treat incontinence in its various forms, including Kegel exercises, electrical stimulation, biofeedback, timed voiding or bladder training, medications, pessaries, implantation of urethral slings to support the urethra, invasive or minimally invasive surgery, catheterization, and other methods and devices.

One additional option for treating urinary incontinence involves periurethral or transurethral injection of a bolus of biocompatible bulk-enhancing or bulking agent into a tissue structure around the urethra including the urethral wall, the bladder neck, bladder suspension ligaments, the urethral sphincter, pelvic ligaments, pelvic floor muscles, fascia, and the like. The injected bulking material adds bulk to the tissue structure to restore and maintains continence, as described, for example, in U.S. Pat. No. 7,014,607 and in U.S. patent application Publication No. 2005/0288639. For example, the bulking agent is injected in or adjacent the urethral sphincter muscle in the area of the urethro-vesical junction, where the urethra extends from the bladder, to increase pressure on and reduce the size of the urethral lumen, providing resistance to the flow of urine. A transurethral injection is made by entering the urethra and penetrating through the urethral wall whereas a periurethral injection is made through the external skin and passing through the tissues surrounding the urethra to dispose the injecting instrument tip in tissue structure in the area of the urethro-vesical junction.

Typical bulking agents include inert Teflon® plastic particles, autologous fat, collagen, and polyhydroxyalkanoate materials. In addition, U.S. Pat. No. 6,702,731 discloses biocompatible, tissue-reactive polymers that bond with tissue to form a bulk polymer in-situ that is biocompatible, elastomeric and non-biodegradable. The bonded polymer remains in place and does not substantially change volume over time.

The periurethral and transurethral injections of such bulking masses are typically done employing a cystoscope to provide direct visualization of the urethra and can be completed on an outpatient basis. However, the physician must be trained in the use of the cystoscope, and its use entails employing relatively expensive cystoscopic equipment in an operating or procedure room and use of anesthesia. The physician must undergo extensive training on how to precisely inject bulking mass with cystoscopic visualization. It would be desirable to provide simplified bulking mass delivery instrumentation to avoid having to use a cystoscope to expose the periurethral space.

A transurethral bulking mass injection procedure employing a specialized instrument adapted to be inserted through the urethra to function as an injection guide and needle stop is disclosed in U.S. patent application Publication No. 2005/0288639 and asserted to obviate the need for the cystoscope and specialized surgical training.

In another option, a detachable balloon can be placed in tissue in the area of the urethro-vesical junction immediately adjacent to the urethra and inflated as disclosed in the above-referenced '699 patent to function as a bulking mass. The inflated balloon provides pressure external to the urethra, which causes partial closure of the urethra and reduces unwanted fluid leakage from the bladder through the urethra. Depending upon the target site location and/or physician preference, the balloon delivery system can be used in parallel with a viewing instrument or can be passed through a lumen of a viewing instrument.

For example, in the periurethral delivery, the deflated balloon and balloon delivery instrument are advanced through the patient's tissue parallel to the urethra to the target site for delivery and inflation of the balloon. The desired target site and correct positioning of the assembly are observed using a viewing instrument passed through, for example, the urethra. In the transurethral delivery, the deflated balloon and balloon delivery instrument are inserted through the lumen of a viewing instrument such as a cystoscope, and the assembly is advanced through the urethral wall to the target site.

In still another treatment option disclosed in U.S. Pat. No. 6,976,492 and in U.S. patent application Publication No. 2005/0288544, thermal heating or cooling energy is applied to induce controlled shrinkage or contraction of a support tissue structure, typically being a collagenated tissue such as fascia, ligament, or the like. For treatment of urinary incontinence, the tissue structure is responsible in some manner for control of urination or for supporting such a tissue, e.g. the urethral sphincter.

Disclosed systems include a urethral guide having a distal balloon adapted to be inserted in the urethra to dispose the balloon in the bladder for inflation therein and a vaginal guide adapted to be inserted into the vagina. The urethral and vaginal guides include treatment delivery surfaces, e.g., RF electrodes or other heating or cooling surfaces, that face one another so that heating energy or cooling is concentrated in the tissue structure disposed therebetween.

A urethral measurement assembly facilitates registration of a treatment delivery surface with a fractional location along the urethral axis, such as the mid-point of the urethra. Ideally, the physician will have some freedom to move a treatment probe manually as desired to achieve the best thermal contact, electrical contact, ergonomic fit to the patient, or the like, while maintaining registration with sufficient tolerances or within an acceptable registration region. The registration region may be established so as to avoid inadvertent damage to nerves or other tissues, which may result from treatments outside, and particularly beyond the desired axial range of, the registration region.

### SUMMARY

The present invention involves delivery methods and instrumentation enabling transvaginal or transperineal (for males) introduction of one or more bolus of a bulking agent or delivery and inflation of an inflatable balloon into a target tissue structure as a bulking mass or masses alongside the urethra effective to at least partially constrict the urethral lumen to treat urinary incontinence. A similar approach is also available for treatment of anal or fecal incontinence by introducing transperineally the bulking agent or balloon near or under the rectum to approximate the ano-rectal angle.

The instrumentation and method of transvaginally delivering a bulking mass into a target tissue structure between the vaginal and urethral walls to constrict the urethra and alleviate incontinence comprises urging a selected portion of the tissue structure between the vaginal and urethral walls into the vaginal cavity as a target structure and passing a bulking mass through the vagina wall and into the target structure.

The transvaginal delivery methods and instrumentation are optimized through the use of specialized vaginal and urethral probes (or transperineal probe) that isolate the target tissue structure between the vaginal and urethral walls and bulking mass delivery instruments introduced through the vaginal probe (or transperineal probe). A transperineal probe can also be used for fecal incontinence bulking to improve continence.

In one approach, the urethral and vaginal probes have elongated and substantially tubular probe bodies, the probe bodies extending axially from probe proximal ends to respective urethral and vaginal probe distal ends. The probe bodies are shaped in a complementary fashion intermediate the probe proximal and distal ends such that the target tissue structure is displaced into a portion of a vaginal probe lumen or a depression or window comprising a recess in the outer surface of the vaginal probe. The urethral probe may comprise a displacement mechanism that extends laterally to the urethral probe axis. The displacement mechanism may be fixed in configuration or deployable laterally from an introduction position to a deployment position urging the target tissue structure into the urethral probe recess. The vaginal probe is preferably configured to enable passage of the distal end of a bulking mass delivery instrument through the probe lumen and into the displaced tissue structure.

In use, the urethral and vaginal probes are positioned and drawn together to urge the target tissue structure into or toward the vaginal probe lumen. One or more bulking mass delivery instrument is inserted through the vaginal probe lumen to dispose the instrument distal end into the target tissue structure. The balloon is delivered and inflated in or the bulking agent is delivered in the target tissue structure, and the instrument and probes are withdrawn.

The probe proximal ends are preferably coupled together by a handle assembly to enable the simultaneous advancement of the probe distal ends and probe bodies substantially side-by-side into the vagina and urethra. The coupling of the probe proximal ends enables movement of the probe bodies substantially laterally to the probe body axes to apply pressure to the tissue structures along the urethra. Preferably, the handle assembly facilitates axial introduction of the delivery instruments through the vaginal probe and lateral deployment and retraction of a deployable displacement mechanism of the urethral probe.

The use of the transvaginal delivery instrumentation to effect transvaginal delivery of the bulking mass may be of particular interest to gynecologists who are accustomed to the vaginal anatomy and transvaginal procedures. The training in the use of a cystoscope is also obviated.

### BRIEF DESCRIPTION OF THE DRAWINGS

FIG. 1 is a fragmentary mid-sagittal cross-sectional view of the pelvic region illustrating the disposition of the urethra, bladder and vagina together with neighboring organs in a healthy woman;

FIG. 2 is a fragmentary transverse view generally as seen along the line 2-2 of FIG. 1, illustrating the pubic bone, the bladder, the urethra, the arcus tendineus fascia, the endopelvic fascia and the pubourethral ligaments;

FIG. 3 is a fragmentary mid-sagittal cross-sectional view, similar to FIG. 1, illustrating the positioning of a vaginal probe in the vagina and a urethral probe of the delivery instrumentation in the urethra to displace target tissue structure into or toward the vaginal probe lumen;

FIG. 4 is a fragmentary mid-sagittal cross-sectional view, similar to FIG. 3, illustrating the vaginal probe in partial section and the displaced target tissue structure;

FIG. 5 is an expanded fragmentary mid-sagittal cross-sectional view, similar to FIG. 4, illustrating the vaginal probe in partial section and the displaced target tissue structure with a bulking mass delivery instrument distal section disposed through an instrument guide of the vaginal probe;

FIG. 6 is a side view of at least a distal section of an exemplary vaginal probe in accordance with one embodiment of the invention;

FIG. 7 is a top view of the distal section of the exemplary vaginal probe of FIG. 6 depicting a tissue structure receiving recess and instrument passage ports;

FIG. 8 is a cross-section view taken along lines 8-8 of FIG. 7 of the exemplary vaginal probe of FIGS. 6 and 7;

FIG. 9 is a side view of the distal section of the exemplary vaginal probe of FIG. 6 and depicts bulking mass delivery instruments inserted through the vaginal probe lumen to dispose the instrument distal ends through ports and into the vaginal probe recess for receiving the target tissue structure;

FIG. 10 is a top view of the distal section of the exemplary vaginal probe and a pair of bulking mass delivery instruments having bulking masses loaded into the delivery instrument lumens, the delivery instruments inserted through the vaginal probe lumen to dispose the instrument distal ends through the ports and into the vaginal probe recess for receiving the target tissue structure;

FIG. 12 is a side view of at least a distal section of an exemplary urethral probe in accordance with an embodiment of the invention having a laterally deployable displacement mechanism disposed intermediate the urethral probe proximal and distal ends;

FIG. 11 is a side view of the distal section of a further exemplary urethral probe having a deployable displacement mechanism adapted to be displaced substantially laterally to the axis of the urethral probe;

FIG. 13 is a side view of the distal section of a further urethral probe depicting a displacement mechanism comprising an inflatable balloon adapted to be displaced substantially laterally to the axis of the urethral probe;

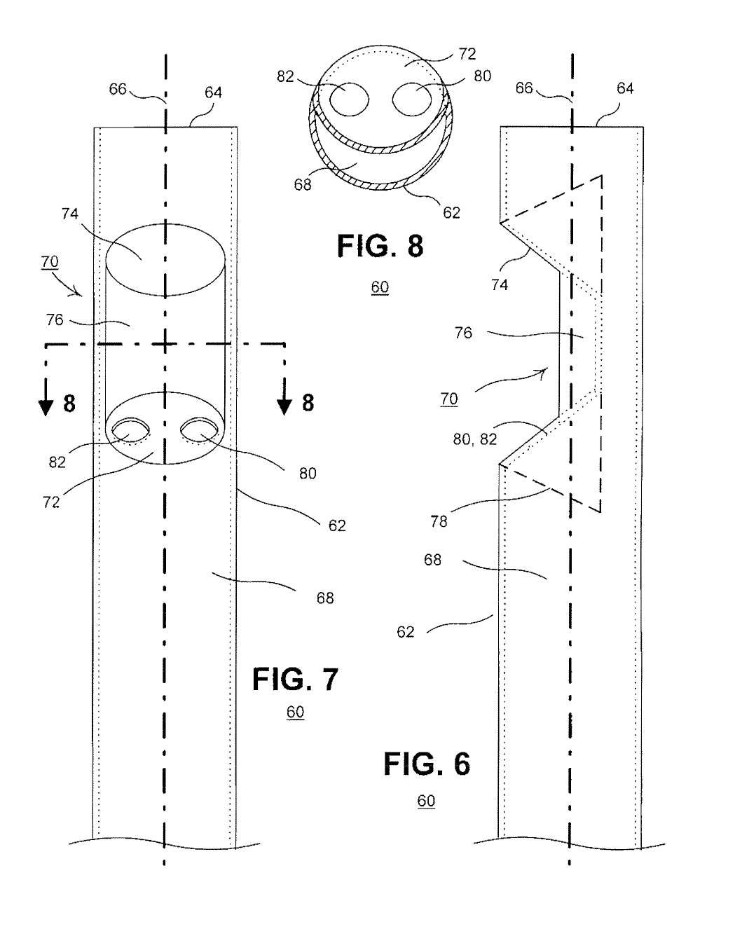

FIG. 14 is a side view of the distal sections of the vaginal probe of FIGS. 6-10 and the urethral probe of FIG. 11, for example, disposed in a patient's respective vagina and urethra with the displacement mechanism oriented and deployed to urge target tissue into the vaginal probe recess and the delivery instruments poised within the vaginal probe lumen to be passed through the probe ports and intro the displaced target tissue structure;

FIG. 15 is a side view of the distal sections of the vaginal probe of FIGS. 6-10 and the urethral probe of FIG. 11, for example, disposed in a patient's respective vagina and urethra with the displacement mechanism oriented and deployed to urge target tissue into the vaginal probe recess and the delivery instrument tips passed through the probe ports and intro the displaced target tissue structure;

FIG. 16 is a side view of the distal sections of the vaginal probe of FIGS. 6-10 and the urethral probe of FIG. 11, for example, disposed in a patient's respective vagina and urethra with the displacement mechanism oriented and deployed to urge target tissue into the vaginal probe recess, the delivery instruments passed through the probe ports, and the bulking masses disposed in the displaced target tissue structure;

FIG. 17 is a cross-section view taken along lines 17-17 in FIG. 16 depicting the displacement mechanism oriented and deployed to urge target tissue into the vaginal probe recess and the bulking masses disposed in the displaced target tissue structure; and

FIG. 18 is a cross-section schematic view of the dispensed bulking masses disposed in the target tissue structure relative to the vaginal and urethral walls following removal of the delivery instrumentation.

The drawing figures are not necessarily to scale

### DETAILED DESCRIPTION OF PREFERRED EMBODIMENTS

In the following detailed description, references are made to illustrative embodiments of methods and apparatus for carrying out the invention. It is understood that other embodiments can be utilized without departing from the scope of the invention.

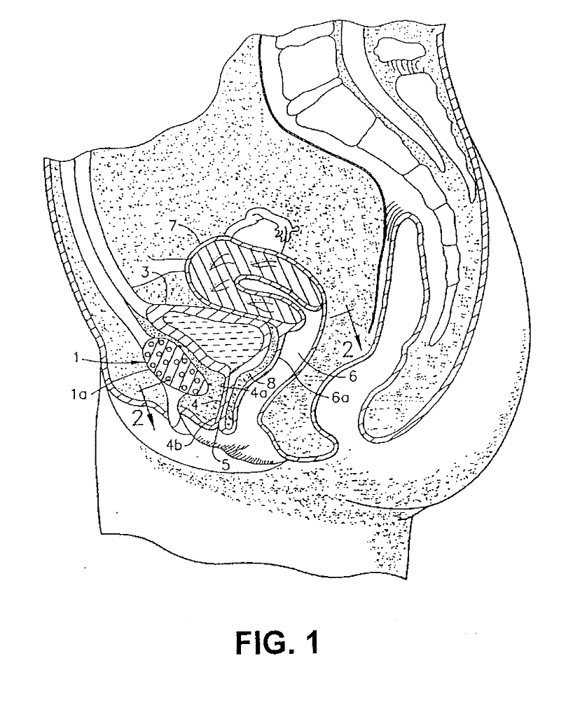

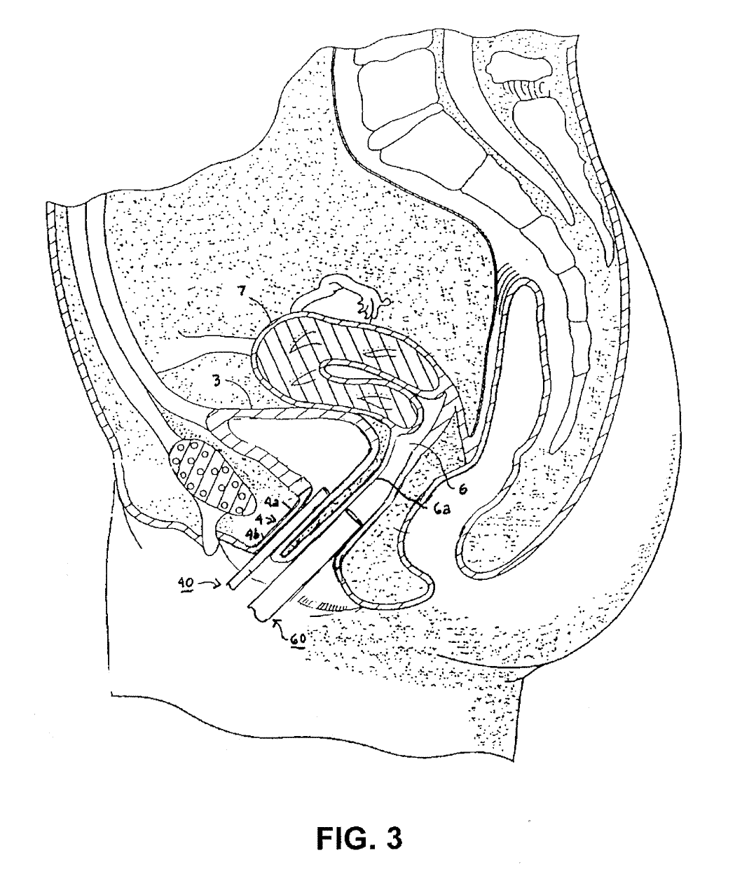

Before describing the methods and apparatus of the present invention reference is directed to the female pelvic anatomy depicted in FIGS. 1-3.

The anterior skeletal bony portions 1a and 1b of the pelvis are joined together by the pubic symphysis 2 to form the pubic bone 1. The bladder 3 and uterus 7 are located superior to and posterior to the pubic bone 1. The urethra 4 extends from the bladder 3 inferiorly to the urinary meatus 5. The vagina 6 extends from the vaginal formix inferiorly alongside the bladder 3 and urethra 4 to the vaginal opening inferior to the urinary meatus 5. The vaginal wall of the vagina 6 surrounds the vaginal cavity. The rectum and anal opening are schematically depicted between the vagina 6 and the sacral bones inferior to the spinal column.

The urethra 4 is defined by a urethral wall encasing the urethral tissue structure including the bladder neck, bladder suspension ligaments, the urethral sphincter, pelvic ligaments, pelvic floor muscles, fascia, and the like. The superior 20% of the length of the urethra 4 constitutes the urethra-vesical junction 4a or bladder neck portion. The inferior 20% of the length of the urethra 4 constitutes the urinary meatus junction 4b with the urinary meatus 5. The urinary sphincter is disposed within and as part of the tissue structure surrounding the intermediate 60% of the length of the urethra 4 between the urethra-vesical junction 4a and the urinary meatus junction 4b. The strength and patency of the urinary sphincter is believed to be of key importance for maintaining continence and can be compromised as the result of prolapse.

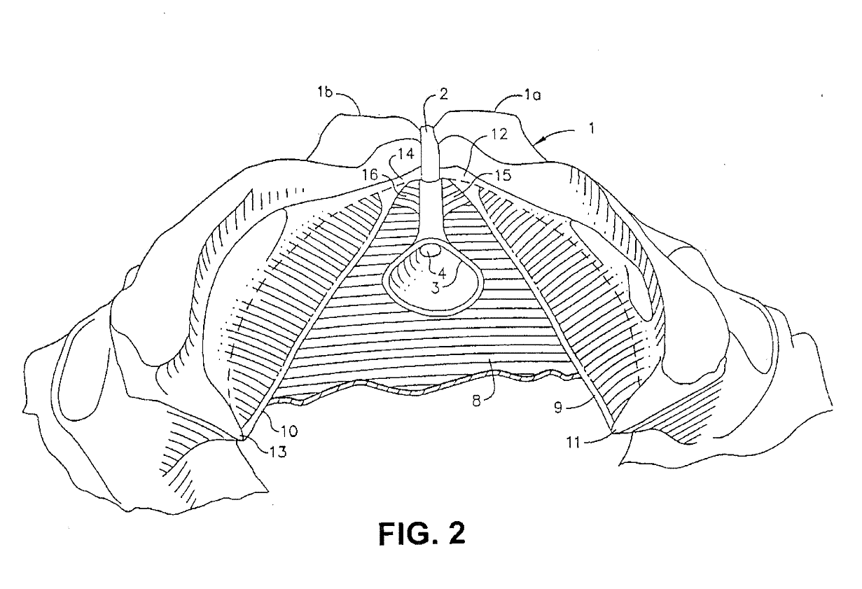

In reference to FIGS. 1 and 2, it has long been understood that female continence is largely a factor of the proper support and stabilization of the bladder 3 and urethra 4 in their normal retropubic state and particularly during coughing, straining and the like. In the healthy, continent female, the urethra 4 and bladder 3 are separated from the extraabdominal area by a hammock-like supportive layer comprising the web of endopelvic fascia 8 and the anterior vaginal wall 6a. As is most clearly shown in FIG. 2, the web of endopelvic fascia 8 is attached to the arcus tendineus fascia pelvis 9 at the right side of the pelvis (as viewed in FIG. 2) and to the arcus tendineus fascia pelvis 10 on the left side of the pelvis (as viewed in FIG. 2). The arcus tendineus fascia pelvis 9 extends from the ischial spine 11 to its insertion in the pubic bone portion 1a at 12. Similarly, the arcus tendineus fascia pelvis 10 extends from the ischial spine 13 to the insertion of the arcus tendineus fascia pelvis in the pelvic bone portion 1b, at 14.

The urethra 4 is additionally supported by a pair of pubourethral ligaments 15 and 16. Pubourethral ligament 15 is attached to the side of urethra 4 and extends forwardly to the pubic bone 1a adjacent the insertion 12 of the arcus tendineus fascia pelvis 9. In a similar fashion, the pubourethral ligament 16 extends from the opposite side of the urethra 4 to the pubic bone 1b adjacent the insertion 14 of the arcus tendineus fascia pelvis 10. The attachment of the pubourethral ligaments to the sides of urethra 4 are located at the above-noted intermediate 60 percent of the urethra. From the above, it will be apparent that weakening of the endopelvic fascia 8, weakening of the anterior vaginal wall 6a, weakening of the attachments to the pubic bone and stretching of the pubourethral ligaments 15 and 16 can result in urethral hyper-mobility, weakening or slackening of the urethral sphincter, and incontinence.

As described above, one treatment option involves periurethral or transurethral delivery of one or more bulking mass into the tissue structure in the intermediate 60% of the length of the urethra 4 between the urethra-vesical junction 4a and the urinary meatus junction 4b to take up the slack and restore the function of the urethral sphincter. The periurethral delivery is effected by passage of a guide or delivery needle through a skin incision into the target structure. The transurethral delivery is effected by passage of a guide or delivery needle through a cystoscope inserted into the urethra lumen and through the urethral wall into the target structure.

For simplicity of discussion, it will be understood that references herein to the vagina 6 include or more precisely refer to the vaginal cavity. For example, insertion of a vaginal probe into the vagina 6 as described herein more precisely refers to insertion of the vaginal probe into the vaginal cavity, and the vaginal cavity may be referenced as the vagina 6 in certain drawing figures. Similarly, references herein to the urethra 4 include or more precisely refer to the urethral lumen that urine passes through and that is bounded by the urethral wall. Consequently, insertion of a urethral probe into the urethra 4 as described herein more precisely refers to insertion of the urethral probe into the urethral lumen, and the urethral lumen may be referenced as the urethra in certain drawing figures.

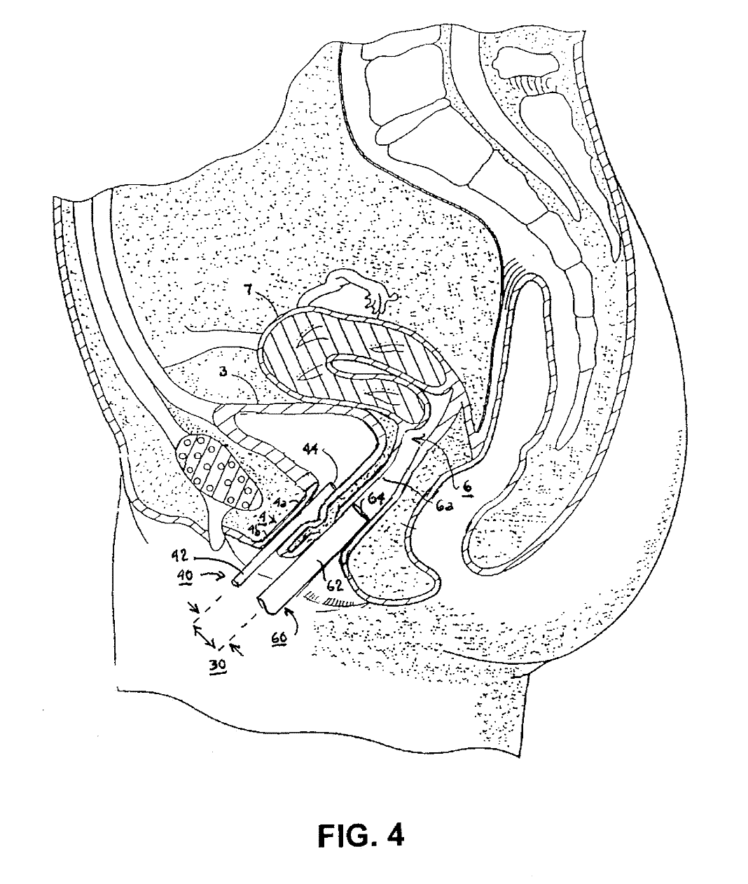

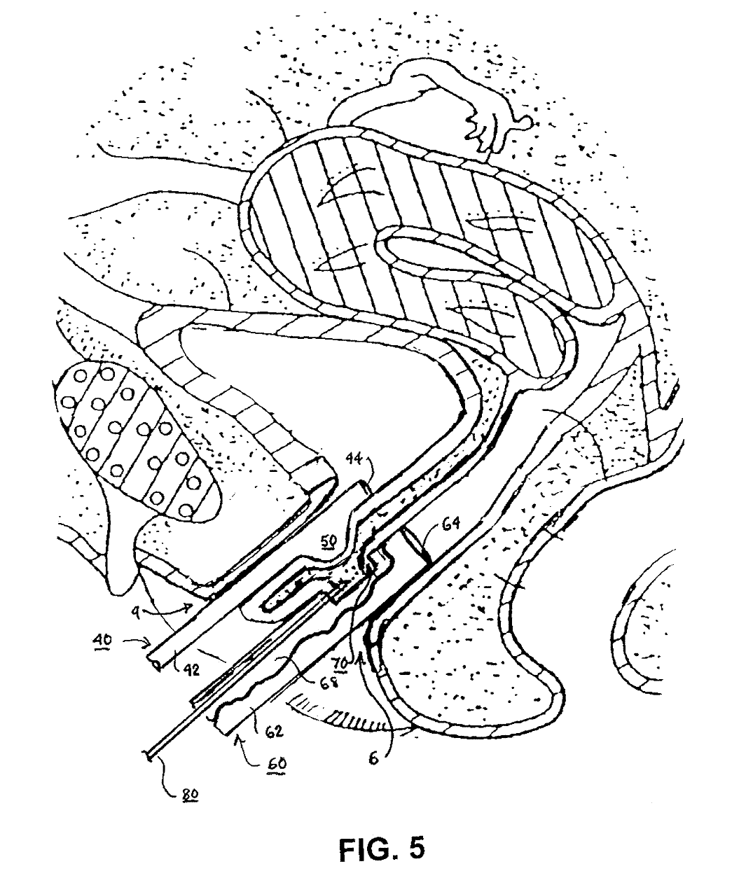

In reference to FIGS. 4 and 5, the present invention involves the transvaginal injection of one or more bulking mass into or alongside the tissue structure within the urethral wall surrounding or alongside the urethral lumen of urethra 4, particularly the urethral sphincter surrounding the intermediate 60% of the length of the urethra 4. The transvaginal delivery is preferably into periurethral tissue lateral to the urethra (at 3 o'clock and 6 o'clock) and the tissue directly between the vagina and urethra. Thus, the location of a target tissue structure between the vaginal and urethral walls includes such locations.

In one preferred embodiment, the delivery is optimized through the use of transvaginal delivery instrumentation 30 schematically shown in FIG. 5 comprising vaginal probe 60 and urethral probe 40 inserted into the respective vagina 6 and urethra 4 to cooperatively isolate the target tissue structure and one or more delivery instrument to introduce the bulking mass through vaginal probe lumen and into the target tissue structure.

It will be understood that the vaginal probe 60 extends between a vaginal probe proximal end and a vaginal probe distal end, and the urethral probe 40 extends between a urethral probe proximal end and a urethral probe distal end. While the probes 40 and 60 are depicted schematically as separate devices, it will be understood that the probe proximal ends may be interconnected with a handle to form the instrumentation 30 that is operable to support the probes 40 and 60 extending substantially in parallel with one another to the probe free ends. It will also be understood that the handle may be operable to increase or decrease the side-by-side, longitudinal spacing or gap between the probes 40 and 60 as shown in FIG. 4 so that pressure may be applied against or withdrawn from the tissue structures including the vaginal and urethral walls and the urethral sphincter therebetween. The coupling of the probe proximal ends enables movement of the probe bodies substantially laterally to the probe body axes to apply pressure to the tissue structures along the urethra. Preferably, the handle assembly facilitates axial introduction of the delivery instruments through the vaginal probe and lateral deployment and retraction of a deployable displacement mechanism of the urethral probe.

At least one of the vaginal probe 60 and the urethral probe 40 is configured having a concave surface or recess extending toward the probe axis that the target tissue structure is urged into when the gap between the probes 40 and 60 is decreased. The other of the vaginal probe 60 and urethral probe 40 is preferably configured with a fixed or deployable displacement mechanism that extends laterally away from the probe axis. In the depicted preferred embodiments, the probe bodies 42 and 62 are shaped in a complementary fashion intermediate the probe proximal ends (not shown) and distal ends 44 and 64 to receive and displace or isolate the target tissue structure between the vaginal and urethral wall into a portion of a vaginal probe lumen or a tissue receiving depression or recess 70 in the wall of the vaginal probe body 62. A laterally extending or extendable tissue displacement mechanism 50 is formed on or deployable from the probe body 42 to urge the target tissue into the tissue receiving recess 70.

The vaginal probe 60 is preferably configured having a guide or template guiding the distal end of a bulking mass delivery instrument 90 through the probe wall into the displaced tissue structure. The guide may simply be a port through the proximal recess wall taking any suitable shape and area that is visible from the probe proximal end via the vaginal probe lumen 68 and sized to receive the distal end of the bulking mass delivery instrument(s) 90.

In use as shown in FIGS. 4 and 5, the urethral and vaginal probes 40 and 60 are positioned and drawn together to urge the target tissue structure into the tissue receiving recess 70. The bulking mass delivery instrument(s) 90 is inserted through the vaginal probe lumen 68 from the probe proximal end to dispose the instrument distal end into the target tissue structure. The balloon is delivered and inflated in or the mass of bulking agent is delivered in the target tissue structure, and the instrument(s) 90 is withdrawn from the vaginal probe lumen 68. The probes 40 and 60 are separated apart and withdrawn from the respective urethra and vagina.

Turning to FIGS. 6-10, one possible shape of the tissue structure receiving recess 70 is depicted in the distal section of an exemplary vaginal probe 60. The recess 70 extends into the probe lumen 68 toward the probe axis 66. The recess 70 may take a variety of forms, having a concave curved recess wall 76 as shown or a flat recess wall. Similarly, the recess proximal and distal ends 72 and 74 may be flat as depicted or curved and may extend obliquely toward probe axis 66 as shown or substantially laterally or perpendicular to the probe axis 66. The volume of the recess 70 may be increased or decreased by adjusting the relative length, width and depth of the recess 70 or by slanting the distal end 74 toward the probe distal end 64 and/or the proximal end 72 toward the probe proximal end as shown in broken lines in FIG. 6. The diameter of the vaginal probe body 62 may be selected to dilate the vagina to the extent considered optimal to perform the procedure without undue discomfort to the patient. A single port or ports 80, 82 are formed through recess proximal end 72.

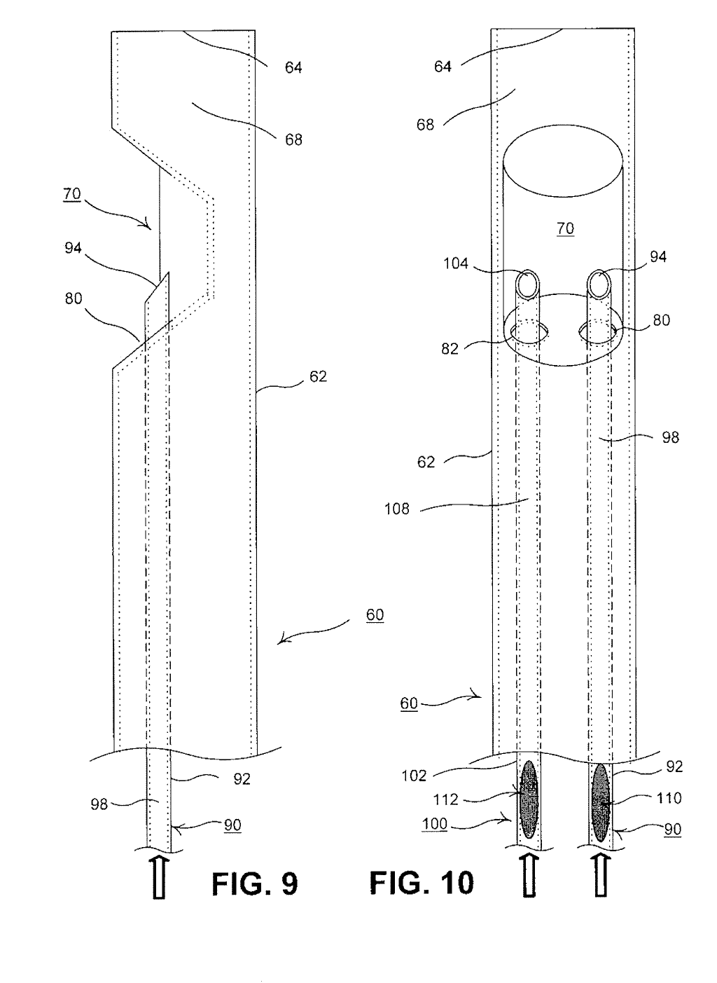

The introduction of distal sections of bulking mass delivery instruments 90 and 100 through the vaginal probe lumen to dispose the instrument distal ends 94 and 104 through the respective ports 80 and 82 and into the tissue receiving recess 70 is depicted in FIGS. 9 and 10. The bulking mass delivery instruments 90 and 100 may comprise elongated delivery instrument bodies 92 and 102 enclosing delivery lumens 98 and 108, respectively, extending from delivery instrument proximal ends (not shown) and delivery instrument distal ends 94 and 104, respectively. The delivery instrument distal ends 94 and 104 are preferably configured to facilitate penetration into the target tissue mass urged into the tissue receiving recess 70, e.g., with slanted needle ends as depicted. The delivery instrument lumens 98 and 108 are open or have distal lumen end openings at the respective delivery instrument distal ends 94 and 104. In FIG. 10, tissue bulking masses 110 and 112 are depicted disposed in the delivery instrument lumens 98 and 108, respectively,

In use, the delivery instruments 90 and 100 are advanced distally as indicated by the arrow in FIG. 9 through the vaginal probe lumen 68 through the respective ports 80 and 82 to dispose the delivery instrument distal ends 94 and 104 in the target tissue mass. The tissue bulking masses 110 and 112 are then advanced distally as indicated by the arrow in FIG. 10 through the delivery instrument lumens 98 and 108, respectively, out of the distal lumen end openings and into the target tissue structure.

Possible configurations of the urethral probe 40 are depicted in FIGS. 11-13. In each case, the urethral probe 40 comprises an elongated probe body 42 extending from a urethral probe proximal end (not shown) to a urethral probe distal end 44 and a probe axis 46.

The urethral probe 40 of FIG. 11 has a displacement mechanism 50 disposed intermediate the urethral probe proximal end and the urethral probe distal end 44 that extends substantially laterally to the urethral probe axis 46 to a pressure applying surface 54. The urethral probe body 42 and the displacement mechanism body 52 may be solid and formed integrally.

In use, the urethral probe 40 and the vaginal probe 60 are advanced into the respective urethra and vagina while spaced apart. The urethral probe body 42 may be rotated 90° or 180° or the like as depicted by the arrow to dispose the displacement mechanism 50 away from the tissue receiving recess 70 during such advancement. Then, the urethral probe body 42 may be rotated back 90° or 180° or the like to dispose the displacement mechanism 50 in alignment with the tissue receiving recess 70.

The vaginal and urethral probes 40 and 60 are brought together to urge the target tissue structure into the tissue receiving recess 70 while the tissue bulking masses 110 and 112 are advanced distally through the delivery instrument lumens 98 and 108, respectively, out of the distal lumen end openings and into the target tissue mass. The urethral probe 40 and the vaginal probe 60 are then separated apart to be withdrawn from the respective urethra and vagina. Again, the urethral probe body 42 may be rotated 90° or 180° or the like to dispose the displacement mechanism 50 away from the tissue receiving recess 70 during such withdrawal.

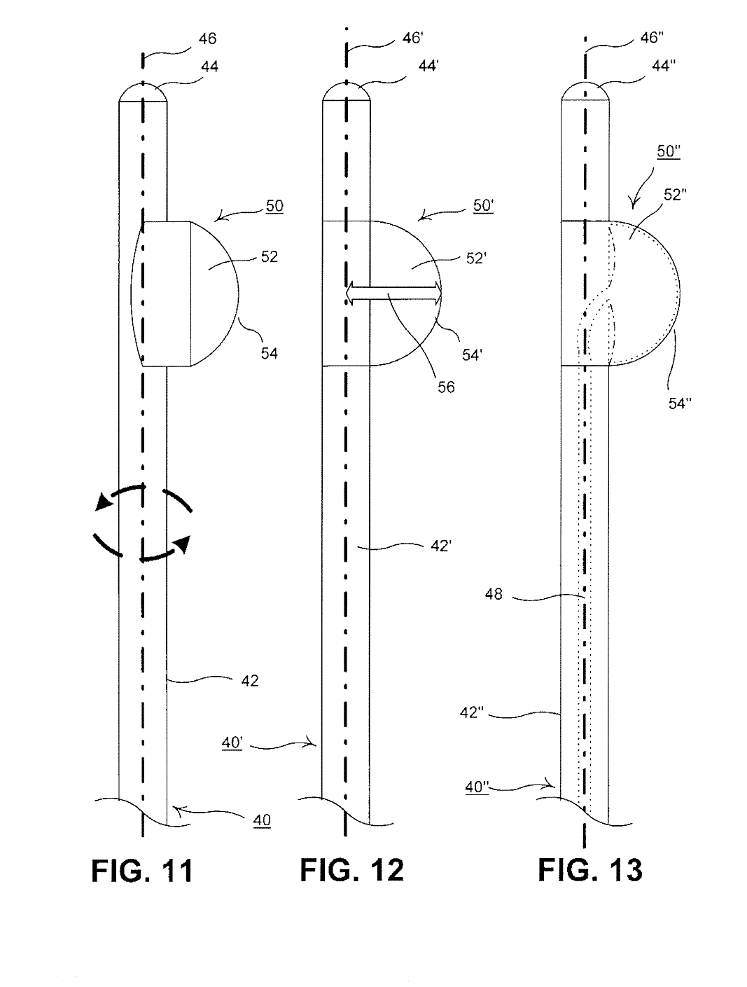

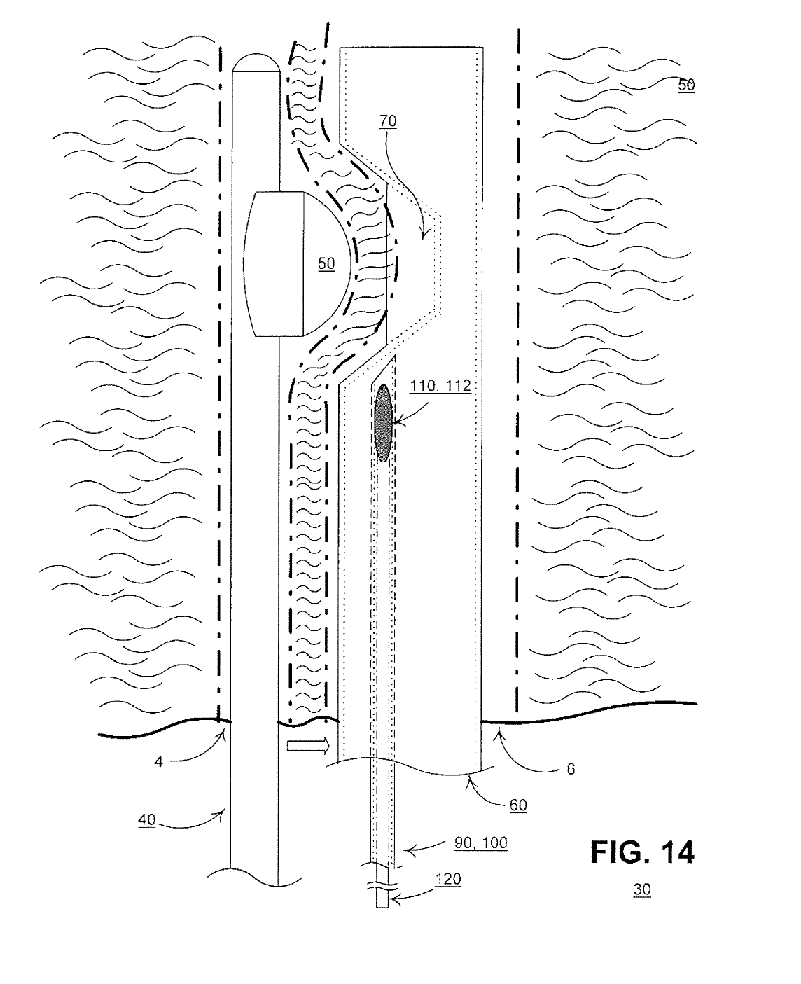

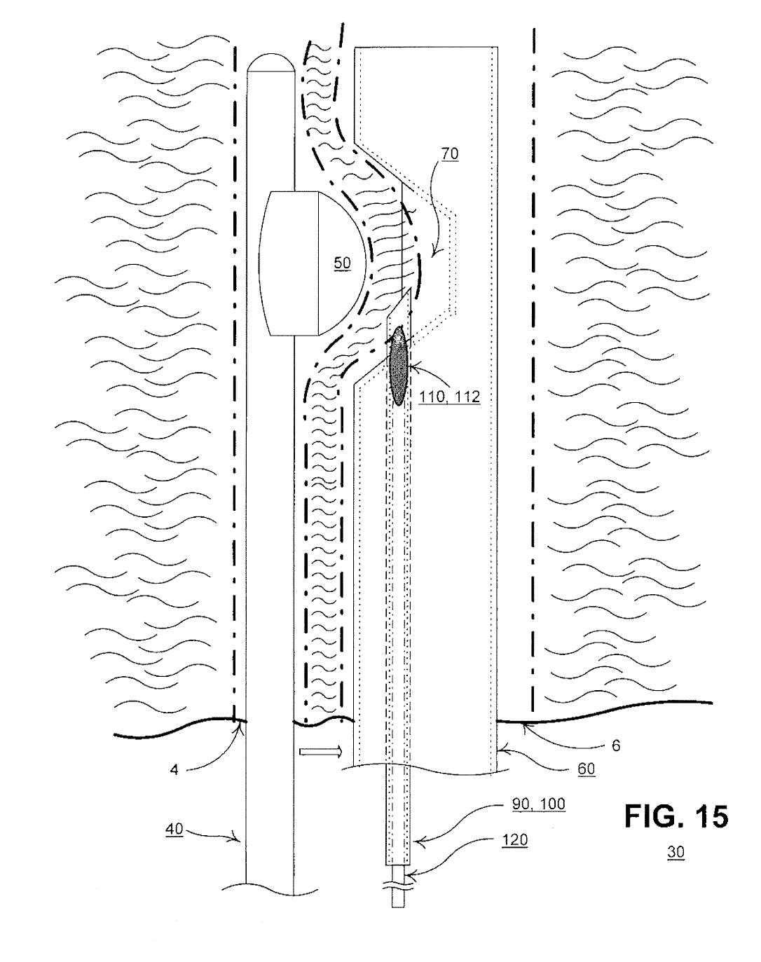

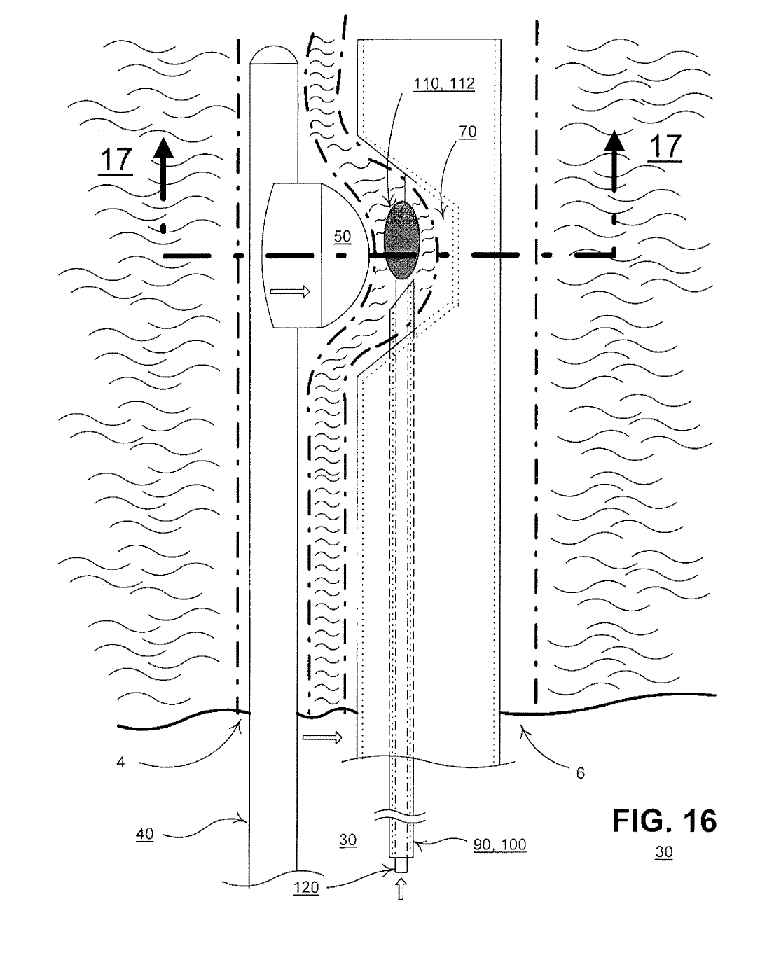

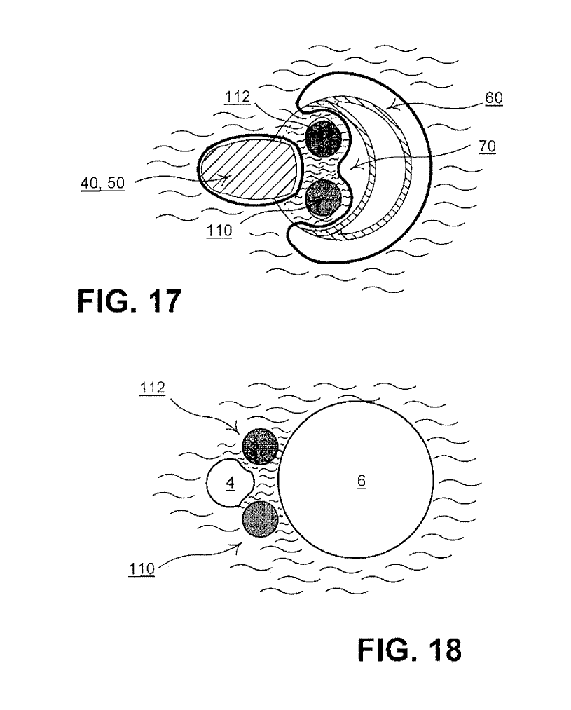

The urethral probe 40′ of FIG. 12 has a displacement mechanism 50′ disposed intermediate the urethral probe proximal end and the urethral probe distal end 44 that is extendable substantially laterally to the urethral probe axis 46 to a pressure applying surface 54′. In this embodiment, a mechanically or electrically activated deployment mechanism 52′ is formed within the displacement mechanism that may be selectively activated from the probe proximal end to extend the pressure applying surface 54′ away from the probe axis 46 to urge target tissue structure into the tissue receiving recess 70 and to retract the pressure applying surface toward the probe axis during urethral probe introduction and withdrawal. The deployment mechanism 52′ may take any form, e.g., a scissor jack or piston or solenoid mechanism having a control extending through the probe body to the handle at the urethral probe proximal end acting on a rigid or resilient pressure applying surface.

The urethral probe 40″ of FIG. 13 has a displacement mechanism 50″ disposed intermediate the urethral probe proximal end and the urethral probe distal end 44 that comprises an inflatable balloon 56 having a pressure applying surface 54″ surrounding the balloon chamber 52″. The balloon chamber 52″ is in communication with an inflation/deflation lumen 48 of the probe body 42 to introduce fluid to inflate the balloon 56 and to withdraw fluid to deflate the balloon 56. The balloon 56 is deflated during introduction and withdrawal of the urethral probe 40″ into and from the urethra and inflated to extend the pressure applying surface 54″ away from the probe axis 46 to urge target tissue structure into the tissue receiving recess 70. The balloon wall may be resilient and compliant such that the inflated balloon volume is variable depending on the amount of fluid introduced through probe lumen 48. Or the balloon wall may be non-resilient such the balloon volume and the shape of the pressure applying surface 54″ are fixed when inflated.

Certain of the above-described steps of implanting two tissue bulking masses 110 and 112 employing the exemplary transvaginal delivery instrumentation are depicted in FIGS. 14-17. It will be understood that the delivery instruments 90, 100 may comprise push rods 120 extending proximally of the delivery instrument proximal ends for pushing tissue bulking masses 110 and 112 through the delivery instrument lumens and out of the distal lumen end opening. In the case that the tissue bulking masses comprise inflatable balloons, it will also be understood that the push rods 120 may also comprise inflation lumens for inflating balloons 110 and 112 before releasing them in the target tissue structure.

In FIG. 14, the distal sections of the vaginal probe 60 and the urethral probe 40, for example, are disposed in a patient's respective vagina 6 and urethra 4 with the displacement mechanism 50 oriented and deployed to urge target tissue into the vaginal probe recess 70. The delivery instruments 90 and 100 are poised within the vaginal probe lumen to be passed through the probe ports and into the displaced target tissue structure. In FIG. 15, the delivery instruments 90 and 100 are advanced within the vaginal probe lumen through the probe ports and into the displaced target tissue structure. In FIGS. 16 and 17, the push rods 120 are advanced distally through the delivery instrument lumens, thereby pushing the tissue bulking masses 110 and 112 into the displaced target tissue structure.

FIG. 18 illustrates the dispensed tissue bulking masses disposed in the target tissue structure relative to the vaginal and urethral walls following removal of the delivery instrumentation. The transvaginal delivery is preferably into periurethral tissue lateral to the urethra (at 3 o'clock and 6 o'clock and the tissue directly between the vagina and urethra. It will be understood that any of the above-described biocompatible tissue bulking agents and inflatable balloons known in the art may be dispensed into the target tissue structure employing the transvaginal delivery methods and instrumentation of the present invention.

It will be understood that the above-described vaginal probe 50 may be modified to have a recess 70 that comprises a window cut out of an arcuate section of the probe wall 62 rather than a depression into the probe wall 62 intermediate the proximal and distal ends, the window eliminating the recess proximal and distal ends 72 and 74 and the concave curved recess wall 76. In this variation, the bulking mass delivery instrument(s) 90 can be introduced through the vaginal probe lumen 68 directly into the target tissue structure displaced into the vaginal probe lumen 68.

All patents and publications referenced herein are hereby incorporated by reference in their entireties.

It will be understood that certain of the above-described structures, functions and operations of the above-described preferred embodiments are not necessary to practice the present invention and are included in the description simply for completeness of an exemplary embodiment or embodiments. It will also be understood that there may be other structures, functions and operations ancillary to the typical surgical procedures that are not disclosed and are not necessary to the practice of the present invention.

In addition, it will be understood that specifically described structures, functions and operations set forth in the above-referenced patents can be practiced in conjunction with the present invention, but they are not essential to its practice.

It is therefore to be understood, that within the scope of the appended claims, the invention may be practiced otherwise than as specifically described without actually departing from the spirit and scope of the present invention.

1. Transperineal delivery instrumentation for delivering a bulking mass into a target tissue structure to constrict the lumen and alleviate incontinence in a patient comprising:
means for urging a selected portion of the tissue structure between the lumen and tissue walls toward the lumen cavity as a target structure; and
means for passing a bulking mass through the lumen cavity and tissue wall and into the target structure.

2. The transperineal delivery instrumentation of claim 1, wherein the bulking mass comprises one of a balloon inflatable within tissue and a bulking agent.

3. The transperineal delivery instrumentation of claim 2, wherein the bulking agent comprises a bolus of one of inert Teflon® plastic particles or autologous or fat or collagen or polyhydroxyalkanoate material or a biocompatible tissue-reactive polymer that bonds with tissue to form a bulk polymer in-situ.

4. Transvaginal delivery instrumentation for delivering a bulking mass into a target tissue structure between the vaginal and urethral walls to constrict the urethra and alleviate incontinence comprising:
a vaginal probe having a vaginal probe lumen adapted to be introduced into the vagina;
means for urging a selected portion of the tissue structure between the vaginal and urethral walls toward the vaginal probe lumen as a target structure; and
a delivery instrument containing a bulking mass adapted to be advanced through the vaginal probe lumen and vaginal wall into the target structure.

5. The transvaginal delivery instrumentation of claim 4, wherein the urging means comprises:
a urethral probe adapted to be introduced into the urethra; and
a laterally extending or extendable tissue displacement mechanism adapted to urge the target structure toward the vaginal probe.

6. The transvaginal delivery instrumentation of claim 5, wherein the vaginal probe comprises an elongated vaginal probe body extending along a vaginal probe axis between vaginal probe proximal and distal ends having a recess formed in the vaginal probe body extending toward a vaginal probe axis, the recess adapted to receive target structure urged toward the vaginal probe by the tissue displacement mechanism of the urethral probe.

7. The transvaginal delivery instrumentation of claim 6, wherein the delivery instrument is adapted to be advanced through the vaginal probe lumen from the vaginal probe proximal end and into target structure urged into the vaginal probe body recess.

8. The transvaginal delivery instrumentation of claim 5, wherein the urethral and vaginal probes have elongated and substantially tubular probe bodies, the probe bodies extending axially from probe proximal ends to respective urethral and vaginal probe distal ends and shaped in a complementary fashion intermediate the probe proximal and distal ends such that the target tissue structure is displaced into a portion of the vaginal probe lumen

9. The transvaginal delivery instrumentation of claim 5, wherein the vaginal probe comprises an elongated vaginal probe body extending along a vaginal probe axis between vaginal probe proximal and distal ends having a recess formed in the vaginal probe body comprising one of a window or depression in the probe body, the recess adapted to receive target structure urged toward the vaginal probe by the tissue displacement mechanism of the urethral probe.

10. The transvaginal delivery instrumentation of claim 9, wherein the delivery instrument is adapted to be advanced through the vaginal probe lumen from the vaginal probe proximal end and into target structure urged into the vaginal probe body recess.

11. The transvaginal delivery instrumentation of claim 4, wherein the urging means comprises:
a urethral probe adapted to be introduced into the urethra; and wherein:
the urethral and vaginal probes have elongated and substantially tubular probe bodies, the probe bodies extending axially from probe proximal ends to respective urethral and vaginal probe distal ends and shaped in a complementary fashion intermediate the probe proximal and distal ends such that the target tissue structure is displaced into a portion of the vaginal probe lumen

12. The transvaginal delivery instrumentation of claim 4, wherein the bulking mass comprises one of a balloon inflatable within tissue and a bulking agent.

13. The transvaginal delivery instrumentation of claim 12, wherein the bulking agent comprises a bolus of one of inert Teflon® plastic particles or autologous or fat or collagen or polyhydroxyalkanoate material or a biocompatible tissue-reactive polymer that bonds with tissue to form a bulk polymer in-situ.

14. A method of transvaginally delivering a bulking mass into a target tissue structure between the vaginal and urethral walls to constrict the urethra and alleviate incontinence comprising:
urging a selected portion of the tissue structure between the vaginal and urethral walls toward the vagina cavity as a target structure; and
passing a bulking mass through the vaginal cavity and vagina wall and into the target structure.

15. The method of claim 14, wherein the bulking mass comprises one of a balloon inflatable within tissue and a bulking agent.

16. The method of claim 15, wherein the bulking agent comprises a bolus of one of inert Teflon® plastic particles or autologous or fat or collagen or polyhydroxyalkanoate material or a biocompatible tissue-reactive polymer that bonds with tissue to form a bulk polymer in-situ.

17. A method of transvaginally delivering a bulking mass into a target tissue structure between the vaginal and urethral walls to constrict the urethra and alleviate incontinence comprising:
introducing a vaginal probe having a vaginal probe lumen into the vagina;
urging a selected portion of the tissue structure between the vaginal and urethral walls toward the vaginal probe lumen as a target structure; and
passing a bulking mass through the vaginal probe lumen and vaginal wall into the target structure.

18. The method of claim 17, wherein the urging step comprises:
introducing a urethral probe into the urethra; and
manipulating the urethral probe to urge the target structure toward the vaginal probe.

19. The method of claim 17, wherein the bulking mass comprises one of a balloon inflatable within tissue and a bulking agent.

20. The method of claim 19, wherein the bulking agent comprises a bolus of one of inert Teflon® plastic particles or autologous or fat or collagen or polyhydroxyalkanoate material or a biocompatible tissue-reactive polymer that bonds with tissue to form a bulk polymer in-situ.
